(19) United States
(12) Patent Application Publication
(10) Pub. No.: US 2008/0281272 A1
(43) Pub. Date: Nov. 13, 2008

(54) Syringe Drivers

(76) Inventors: Neal Blundred, St Asaph (GB); Mark Dodgson, St Asaph (GB); Kenneth Hall, Stype (GB)

Correspondence Address: CHARLES N. QUINN;FOX ROTHSCHILD LLP, 2000 MARKET STREET, 10TH FLOOR, PHILADELPHIA, PA, 19103, US

(21) Appl. No.: 11/793,935

(22) PCT Filed: Dec. 22, 2005
(86) PCT No.: PCT/GB2005/005027

§ 371 Date: Jul. 1, 2008

(30) Foreign Application Priority Data
Dec. 22, 2004 (GB) 0428048.3

(51) Int. Cl. A61M 5/20

(52) U.S. Cl. 604/134; 604/131

(57) ABSTRACT

A syringe driver (10) comprising retaining means for retaining the body of a syringe, and exerting means (20) for exerting a force to the syringe plunger, and sensing means (52) for sensing the compressive force applied to the syringe plunger in use, wherein the sensing means comprises a pressure sensitive material, such as a quantum tunneling material.

The present invention relates to syringe drivers.

A syringe driver generally comprises a mechanical assembly adapted to engage with a syringe. The mechanical assembly is used to decant a specified amount of liquid, e.g. a drug, from the syringe into a user at a desired rate. As syringe driver technology has developed, certain advantages have been discovered in varying the speed of drug delivery. For example, it is desirable to monitor the backpressure in a syringe, i.e. the pressure of the user's blood against which the drug is being forced, to ensure that the drug delivery rate is compliant with one or more physiological parameters.

One problem with known syringe drivers is that they deliver the liquid e.g. a medication, at a predetermined rate. This state of affairs is normally satisfactory, however certain problems can, from time to time, arise.

For example, the delivery of the drug may cause an adverse reaction resulting in the blood pressure of the patient rising. This can be dangerous or indicate a medical complication. Known syringe drivers do not have means for detecting this situation and will continue to deliver the medication, which may prove catastrophic.

Furthermore, if the intravenous (IV) line becomes obstructed i.e. kinked or blocked (e.g. by clotted blood), known syringe drivers will nonetheless continue to deliver the medication. This will result in a pressure build-up in the IV line, which may cause the obstruction to move or dislodge or the kink to straighten itself. As the obstruction is cleared, medication will be delivered at an abnormally high pressure due to the increase in IV line pressure, which may cause complications e.g. vein rupture or cavitation whereby bubbles are introduced into the bloodstream, which can be fatal.

Our co-pending patent application (GB0421858.2) describes a syringe driver system utilising a motor to drive the syringe, wherein the motor torque (which has been found to be a function of backpressure) is monitored using a modified strain gauge.

An object of the present invention is to provide an improved syringe driver. Ideally, the invention provides a syringe driver capable of monitoring the backpressure in the syringe or IV line and responding to an abnormal or undesirable backpressure condition.

Accordingly, a first aspect of the invention provides a syringe driver comprising retaining means for retaining the body of a syringe, an exerting means for exerting a force to the syringe plunger, and sensing means for sensing the compressive force applied to the syringe plunger in use, wherein the sensing means comprises a pressure sensitive material.

The pressure sensitive material can be of any suitable type. However, it is envisaged that a quantum tunneling material or a piezoceramic material be used whereby an electrical property (e.g. conductivity or dipole moment) changes as the material is strained.

The exerting means may comprise a pad arranged to abut and press against the syringe plunger in use. The sensing means is preferably associated with the pad. In a preferred embodiment of the invention, the sensing means interacts with and senses the force exerted by a part of the syringe drive mechanism during use. Specifically, the sensing means may be connected to a shaft that drives the exerting means to sense either linear displacement thereof (a function of linear stress) or torsion therein (a function of torque).

Preferably, the sensing means is mounted between washers that are fixedly secured to the shaft. Pre-compression may be applied to the sensing means. Biasing means may also be provided between the washers, such as compression springs, to enable the washer spacing to return to a predetermined distance. It is preferable for the sensing means to comprise at least two discs, preferably being mounted between three washers.

The syringe driver can be of any suitable type, although it is envisaged that it will comprise a body for housing the various components, i.e. the retaining means and the exerting means and the sensing means. The body is preferably manufactured of durable materials and is preferably cleanable or sterilisable using solvents or a autoclave. Plastics materials are preferred for the construction of the syringe driver body. The body may also comprise a structural chassis for strength, rigidity and/or durability to which the various components may be affixed.

The retaining means may be of any suitable type, although a channel corresponding to the shape of the syringe body and a clamp for clamping the syringe body to the channel is a possible preferred arrangement. The channel, where provided, may simply comprise a pair of spaced-apart ribs. The clamp, where provided may comprise a sensor for sensing the presence of a syringe and/or correct alignment/clamping thereof. The clamp is preferably biased to a clamping position (e.g. spring loaded) such that it can be retracted by hand and released onto the syringe body when the syringe body is in situ. The channel and hook arrangement, where provided, preferably comprise features, e.g. ribs or nodules, that serve to self-align the syringe with respect to the syringe driver.

The exerting means preferably comprises a linear actuator. Any linear actuator may be used, although a geared worm screw driven by an electric motor is one possible option. Alternatively, a carriage may be driven along a smooth shaft by mounting the carriage on the shaft using canted bearings and by rotating the shaft. A simple friction wheel arrangement or a rack and pinion arrangement may be used. The precise details of the exerting means are not important, although some methods may be advantageous in certain respects, so long as a force is capable of being applied to the syringe plunger.

The exerting means may comprise a pad arranged to abut and press against the syringe plunger in use. The pad may be flat or may have formations thereon for gripping the syringe plunger. Such formations may be clips that engage over the edges of the plunger head.

The sensing means associated with the pad is preferably a quantum tunneling composite (QTC) material. The QTC material has a conductivity that can be altered by applying a compressive force thereto. This effect may be achieved in any suitable manner, e.g. by disposing conductive particles in a non-conductive matrix and spacing the particles sufficiently close that electron tunneling can occur when an electrical potential is applied across the QTC material. Compression of the QTC material may cause the particles to move closer together, thereby increasing the probability of electron tunneling events (which is inversely dependent on the particle spacing) and reducing the effective resistance of the material.

One possible proprietary QTC material suited to the present application is the Peratech™ switch substrate material.

It is preferable to provide a stiffening shim material on one side of the QTC material to improve handling and performance. For example, the material may comprise a sheet of acetate. However, any suitable non-compressive non-conductive material may be used. The stiffener should be of a known thickness. The stiffener provides for better handling of the sensing means and improves performance. The stiffened QTC material is less susceptible to bending resulting in more even surface contacts being achieved.

The syringe drive preferably comprises a control circuit for controlling various functions, including driving the exerting means and monitoring the backpressure. The control circuit preferably comprises a number of integrated circuits and may have a display for displaying the various statuses of the driver.

The control circuit is preferably user-programmable to enable a specific delivery regime to be carried out. The control circuit also, preferably comprises a logic element that serves to modify the delivery regime dependent on the backpressure. In one embodiment, the logic element serves to switch off the exerting means when the back pressure goes above a predetermined point. In another embodiment, the logic element may serve to vary the delivery rate to maintain a constant backpressure. Additionally or alternatively, the logic element may serve to reciprocate the exerting means to try to dislodge any blockage in the IV line using a repetitive pressure build and release regime.

A second aspect of the invention provides a method of driving a syringe comprising the steps of retaining the body of a syringe, exerting a compressive force on the syringe plunger, and sensing the compressive force applied to the syringe plunger in use by sensing a conductivity change in a pressure sensitive material whose conductivity changes as a function of strain.

A preferred embodiment of the invention shall now be described, by way of example only, with reference to the accompanying drawings, in which;

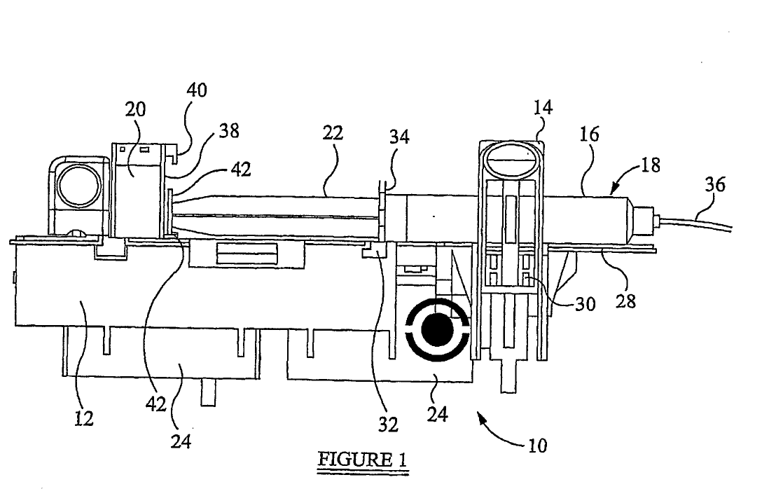

FIG. 1 shows a perspective view of the internal elements of a syringe driver according to the invention;

FIG. 2 shows a perspective top view detail of the exerting means of the invention;

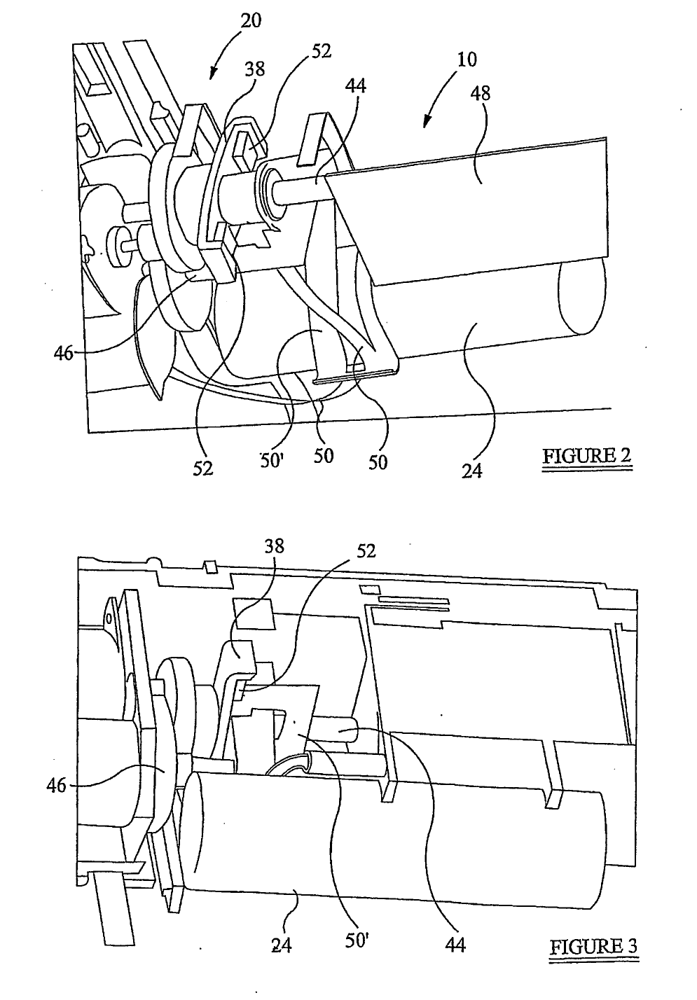

FIG. 3 shows a perspective underside detail of the exerting means of the invention;

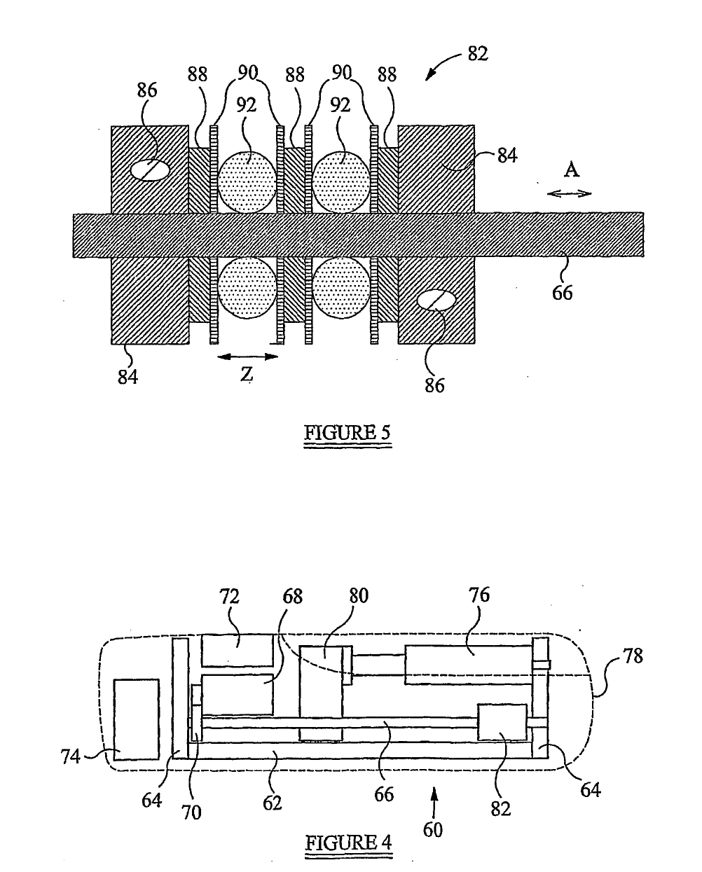

FIG. 4 shows a schematic representation of a syringe driver;

FIG. 5 shows a schematic QTC sensor adapted to sense linear displacement in the drive shaft;

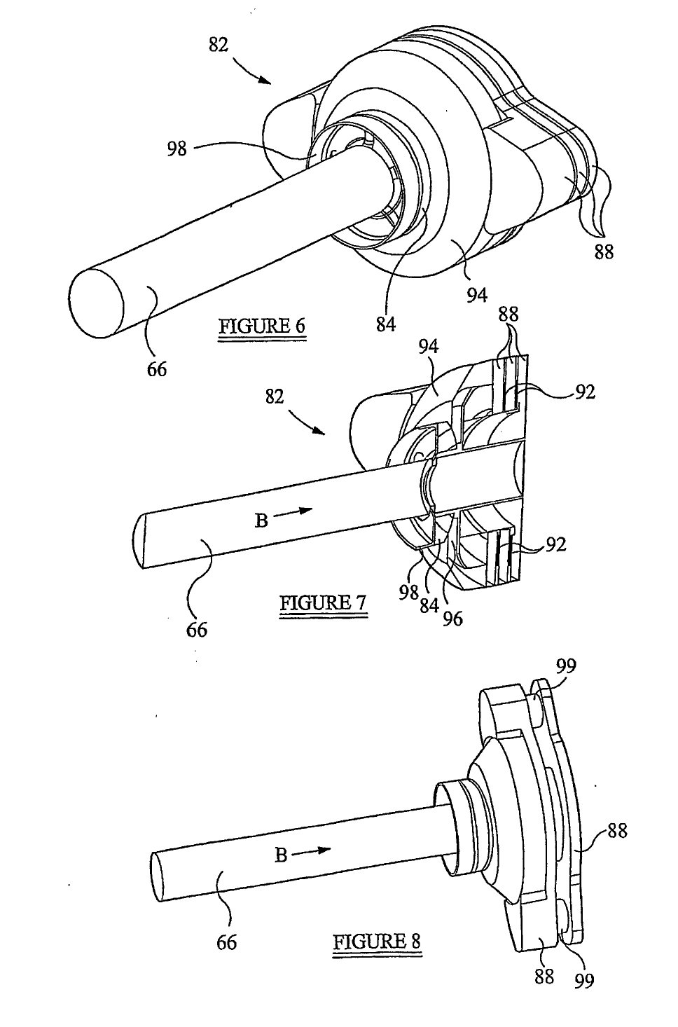

FIG. 6 shows a perspective view of a displacement sensor;

FIG. 7 shows a section of FIG. 6; and

FIG. 8 is a perspective view of a displacement sensor with the middle washer removed to show the location of the compression springs.

Referring now to FIG. 1, the internal elements of a syringe driver 10 are shown comprising chassis 12, a retaining means 14 for retaining the body 16 of a syringe 18, an exerting means 20 for exerting a force onto the plunger 22 of the syringe 18. The exerting means 20 is motor driven using batteries 24. The syringe 18 is connected to an IV line 36. A plastics housing (not shown) is also provided to encase the internal elements and protect them from contamination and damage.

The retaining means 14 comprises a channel 28 in which the syringe 18 sits and a spring-loaded hook 30 that clamps the syringe 18 down into the channel 28. A groove 32 is provided for engaging the tabs 34 on the syringe body 16.

The syringe plunger 22 is pressed against by a pad 38 that has clips 40 for engaging the thumb plate 42 on the end of the syringe plunger 22.

Referring now to FIGS. 2 and 3, the exerting means 20 is shown in greater detail. A motor is used to rotate a shaft 44 via a gearing arrangement 46 which drives a carriage. A pad 38 is arranged to exert a force onto the syringe plunger (not shown). A control circuit 48 is used to control the syringe driver 10. The control circuit 48 is manufactured on a flexible printed circuit board (flexi-PCB) or using conductive ink, such as screen printed using silver ink. Various limbs 50 extend from the control circuit 48 carrying wires connected to sensors, the motor, batteries 24 etc. Each limb 50 is also manufactured of flexi-PCB. One limb 50′ wraps around the pad 38 and is provided with QTC pads 52. Two QTC pads 52 are provided as a double-check measure in case one should fail. The QTC pads 52 are disposed between the pad 38 and the syringe plunger tab 42 in use. Thus, the more force is applied to the syringe plunger 22, the greater the degree of compression present in each QTC pad 52. The compression causes the conductivity of the QTC pad 52 to increase, which increase is detected by the control circuit 48 such that a suitable response can be made.

FIG. 4 shows an alternate syringe driver configuration 60 comprising a chassis 62 that supports end plates 64. The ends of a drive shaft 66 pass through apertures in the end plates 64. The drive shaft 66 is rotated by a motor 68 via a gear assembly 70. A control module 72 controls the operation for the syringe driver 60, which is powered by a rechargeable battery 74. The syringe 76 is supported by a casing 78 and is squeezed by an exerting means 80, which is movable along the drive shaft 66. A sensor 82 detects linear stress in the drive shaft 66, which is proportional to the force exerted by the exerting means 80 on the syringe 76.

FIG. 5 shows a detailed view of the sensor 82. The drive shaft 66 has a pair of collars 84 affixed thereto by way of grub screws 86. Between the collars 84 are located three spaced apart washers 88. Contacts 90 are disposed on faces of the washers 88 to which electrodes (not shown) are attached. Between the contacts are a pair of toroidal QTC sensors 92 approximately 2 mm thick (z). The collars 84 are positioned to place the QTC sensors under a slight baseline compressive force, without which detection of changes would be difficult.

In use, the drive shaft 66 is rotated, which causes the exerting means 80 to press against the syringe plunger 76. This creates a slight linear displacement A in the drive shaft 66, which causes the QTC toroids 92 to compress slightly. The electrical conductivity between the contacts 90 changes as a result, which change is detected by the controller 72. An excessive displacement A is indicative of an increase in backpressure or a fault, which will trigger the syringe driver to shut down and alert the user.

A pair of toroidal QTC sensors 92 and contacts 90 is used as a double safety feature. Thus, if one sensor fails, a backup is always present. Additionally, the outputs of the two sensor pairs can be used to yield an average displacement value.

A further possibility is to fix the position of the middle washer relative to the chassis of the syringe driver. Thus, right hand displacement of the shaft 66 will cause the left hand QTC material 92 to compress, whereas left hand displacement of the shaft 66 will cause the right hand QTC sensor 92 to compress.

The QTC sensors 92 could be replaced by alternative pressure sensitive materials, such as a piezo-ceramic material, a conducting polymer or resistive material.

FIGS. 6 and 7 show a perspective view and a sectional view of the sensor 82, which is positioned at an end of the drive shaft 66.

Spaced apart washers 88 are provided between which discs of QTC material 92 are disposed. A thrust washer 94 bears against an outer washer 88. The thrust washer 94 has a tapered portion 96 that engages with a conical collar 84 affixed by a luck nut 98 to the drive shaft 66. A conical collar 84 is provided to centre the drive shaft with the washers 88.

Movement of the drive shaft 66 in the direction indicated by arrow B causes the collar 84 to press into the thrust washer 94, which exerts a compressive force on the washers 84. The QTC discs 92 are thereby compressed, which gives rise to change in the conductivity of the QTC discs 92.

Finally, FIG. 8 shows the same arrangement, but with the middle washer 88 removed. As can be seen, a pair of compression springs 99 are provided between the outer washers 88 to force them apart. A pre-compression is applied to the QTC discs 92 by the lock nut, however, when the displacement B is returned to zero, it is important that the washer spacing returns to a predetermined extent. The compression springs therefore ensure a desired washer spacing with no linear force applied to the drive shaft 66.

1. A syringe driver (10) comprising retaining means (14) for retaining the body of a syringe, an exerting means (20) arranged to abut and press against a syringe plunger in use, and sensing means (52) for sensing the compressive force applied to the syringe plunger in use, wherein the sensing means comprises a pressure sensitive material and is connected to a shaft (44) that drives the exerting means, characterised in that the sensing means is mounted between washers (88) that are fixedly secured to the shaft and wherein pre-compression is applied to the sensing means, and further comprising biasing means (99) to enable the washer spacing to return to a predetermined distance.

2. A syringe driver as claimed in claim 1 wherein the pressure sensitive material is selected from a quantum tunneling material and a piezoceramic material.

3. A syringe driver as claimed in claim 2 wherein the pressure sensitive material is a quantum tunneling material.

4. A syringe driver as claimed in claim 3 wherein a stiffening shim material is provided on one side of the quantum tunneling material to improve handling and performance.

5. A syringe driver as claimed in claim 4 wherein the stiffening shim material comprises acetate.

6. A syringe driver as claimed in claim 1 wherein the exerting means (20) comprises a pad (38) arranged to abut and press against the syringe plunger in use.

7. (canceled)

8. (canceled)

9. (canceled)

10. (canceled)

11. (canceled)

12. A syringe driver as claimed in claim 1 wherein the biasing means (99) are compression springs provided between the washers.

13. A syringe driver as claimed in claim 1 wherein the sensing means comprises at least two discs (92) mounted between three washers (88).

14. A syringe driver as claimed claim 1 wherein the retaining means (14) comprises a channel (28) corresponding to the shape of the syringe body and a clamp (30) for clamping the syringe body to the channel.

15. (canceled)

16. A syringe driver as claimed in claim 1 wherein the shaft is a geared worm screw (44) driven by an electric motor.

17. A syringe driver as claimed in claim 1 wherein the shaft is a smooth shaft for driving a carriage therealong by mounting the carriage on the shaft using canted bearings and by rotating the shaft.

18. A syringe driver as claimed in claim 5 wherein the quantum tunneling material is Peratech™ switch substrate material.

19. A syringe driver as claimed in claim 1 wherein the syringe driver comprises a control circuit (48) for controlling various functions, including driving the exerting means and monitoring the backpressure.

20. A syringe driver as claimed in claim 19 wherein the control circuit (48) is user-programmable to enable a specific delivery regime to be carried out.

21. A syringe driver as claimed in claim 20 wherein the control circuit (48) comprises a logic element that serves to modify the delivery regime dependent on the backpressure.

22. A syringe driver as claimed in claim 21 wherein the logic element serves to switch off the exerting means (20) when the back pressure goes above a predetermined point.

23. A syringe driver as claimed in claim 21 wherein the logic element serves to vary the delivery rate to maintain a constant backpressure.

24. A syringe driver as claimed in claim 21 wherein the logic element serves to reciprocate the exerting means (20) to try to dislodge any blockage using a repetitive pressure build and release regime.

25. (canceled)
